(19) United States
(12) Patent Application Publication
(10) Pub. No.: US 2023/0320850 A1
(43) Pub. Date: Oct. 12, 2023

(54) ACCESS SHEATH FOR PROSTHETIC CARDIAC VALVE DELIVERY SYSTEMS

(71) Applicant: SHIFAMED HOLDINGS, LLC, Campbell, CA (US)

(72) Inventors: David Jerry SCOTT, San Mateo, CA (US); Noah GOLDSMITH, Campbell, CA (US)

(21) Appl. No.: 18/043,526

(22) PCT Filed: Aug. 27, 2021
(86) PCT No.: PCT/US21/47958

§ 371 Date: Feb. 28, 2023

Related U.S. Application Data
(60) Provisional application No. 63/072,834, filed on Aug. 31, 2020.

(51) Int. Cl. A61F 2/24; A61F 2/95

(52) U.S. Cl. CPC A61F 2/2427; A61F 2/9517

(57) ABSTRACT

Delivery systems for delivering a valve prosthesis into the heart. A delivery system includes an access sheath configured to enter the patient's body and/or heart. The access sheath includes an inner lumen for accommodating one or more delivery catheters carrying one or more parts of the valve prosthesis. The access sheath may include a handle for controlling interchangeable access to the inner lumen of the access sheath. The handle may be configured to provide a hemostatic seal between an outer environment and the inner lumen of the access sheath. In some cases, a bending component is configured to bend the access sheath to facilitate entry of the access sheath into the patient's body.

### INCORPORATION BY REFERENCE

This application claims priority to U.S. Provisional Application No. 63/072,834, titled “ACCESS SHEATH FOR PROSTHETIC CARDIAC VALVE DELIVERY SYSTEMS,” filed Aug. 31, 2020, the entirety of which is incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Blood flow between heart chambers is regulated by native valves—the mitral valve, the aortic valve, the pulmonary valve, and the tricuspid valve. Each of these valves is a passive one-way valve that opens and closes in response to differential pressures. Patients with valvular disease have abnormal anatomy and/or function of at least one valve. For example, a valve may suffer from insufficiency, also referred to as regurgitation, when the valve does not fully close, thereby allowing blood to flow retrograde. Valve stenosis can cause a valve to fail to open properly. Other diseases may also lead to dysfunction of the valves.

The mitral valve, for example, sits between the left atrium and the left ventricle and, when functioning properly, allows blood to flow from the left atrium to the left ventricle while preventing backflow or regurgitation in the reverse direction. Native valve leaflets of a diseased mitral valve, however, do not fully prolapse, causing the patient to experience regurgitation.

While medications may be used to treat diseased native valves, the defective valve often needs to be repaired or replaced at some point during the patient's lifetime. Existing prosthetic valves and surgical repair and/or replacement procedures may have increased risks, limited lifespans, and/or are highly invasive. Some less invasive transcatheter options are available, but most are not ideal. A major limitation of existing transcatheter mitral valve implantation methods, for example, is that a large number of catheters are required to be interchanged during the procedure, and maintaining hemostasis throughout the process can be challenging.

Thus, a new valve delivery system or method that overcomes some or all of these deficiencies is desired.

### SUMMARY

Described herein are systems, devices, and methods related to delivering heart valve prostheses into a patient's heart. A delivery system can include an access sheath that is configured to provide access into the patient's body and/or heart. The access sheath includes an inner lumen for accommodating one or more delivery catheters carrying one or more parts of the valve prosthesis. A handle is configured to provide interchangeable access for the one or more delivery catheters into the inner lumen of the access sheath.

According to some aspects, a delivery system for delivering a valve prosthesis comprises: one or more catheters holding at least a portion of the valve prosthesis therein; an access sheath having an inner lumen for accommodating the one or more catheters; and a handle coupled to a proximal end of the access sheath, the handle comprising a seal configured to accommodate the one or more catheters therethrough and to provide a hemostatic seal between an outer environment and the inner lumen of the access sheath.

In this aspect, the seal can be a disk-shaped insert having a first opening and a second opening, wherein the first opening has a first diameter that is configured to accept and seal with a first catheter, wherein the second opening has a second diameter that is configured to accept and seal with a second catheter.

According to some aspects, a delivery system for delivering a valve prosthesis comprises: one or more catheters holding at least a portion of the valve prosthesis therein; an access sheath having an inner lumen for accommodating the one or more catheters; a handle coupled to a proximal end of the access sheath, the handle configured to provide access for the one or more catheters distally through the access sheath; and a sheath bending component positioned around a circumference of the access sheath, the sheath bending component configured to controllably bend the access sheath.

In this aspect, the sheath bending component can include a hinge and a cable, wherein tensioning of the cable causes the hinge to bend, thereby causing the access sheath to bend accordingly.

These and other aspects are described herein.

### BRIEF DESCRIPTION OF THE DRAWINGS

Novel features of embodiments described herein are set forth with particularity in the appended claims. A better understanding of the features and advantages of the embodiments may be obtained by reference to the following detailed description that sets forth illustrative embodiments and the accompanying drawings.

FIGS. 1A and 1B illustrate an exemplary trans-atrial method of delivering a cardiac valve.

FIG. 2 illustrates an exemplary trans-septal or trans-femoral approach using a delivery sheath to deliver a cardiac valve.

FIGS. 3A-3C illustrate an exemplary surgical delivery system with an access sheath.

FIG. 4 illustrates an exemplary hub of an access sheath.

FIGS. 5A-5B illustrate another exemplary hub of an access sheath.

FIGS. 6A-6F illustrate an exemplary use of the hub and access sheath of FIGS. 5A-5B.

FIGS. 7A-7C illustrate an exemplary seal assembly that may be used as a variation of the rotatable knob assembly of FIG. 3B.

FIGS. 8A and 8B illustrate an exemplary variation of the seal assembly of FIGS. 7A-7C.

FIGS. 9A-9E illustrate an exemplary surgical delivery system having a sheath bending component.

### DETAILED DESCRIPTION

Described herein are devices and methods for use in delivering a cardiac valve, for example during a mitral valve replacement.

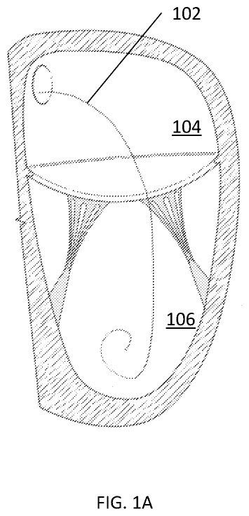

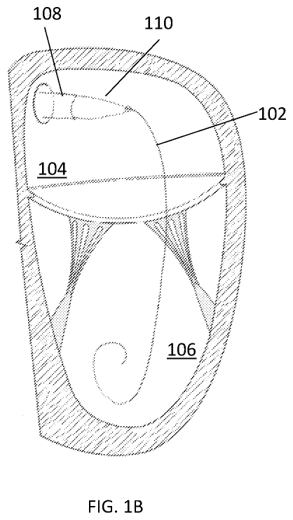

FIGS. 1A-B show a trans-atrial method of delivering a cardiac valve. At FIG. 1A, a trans-atrial puncture is made. A guidewire 102 is then routed through the puncture site and left either in a left atrium 104 or across the mitral valve into a left ventricle 106. At FIG. 1B, an access sheath 108 (with an inner dilator 110) is tracked over the guidewire 102 until the distal end of the access sheath 108 protrudes into the left atrium 104. The guidewire 102 and inner dilator 110 are then removed from the access sheath 108, and catheter(s) holding the replacement valve and/or anchor can be delivered therethrough (e.g., via a valve and/or anchor delivery system).

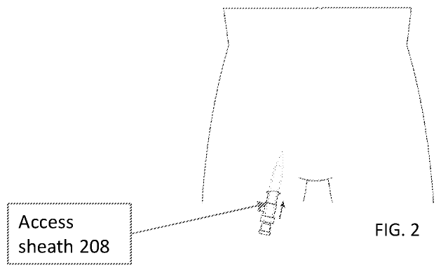

In some embodiments, referring to FIG. 2, a trans-septal or trans-femoral approach can instead be used to deliver a cardiac valve. In such an approach, an access sheath 208 can be inserted into the femoral vein near the groin (with the handle remaining outside of the body). Further, once in place, the inner diameter of the access sheath 208 can be used to accommodate passage of other delivery device components therethrough.

In still other embodiments, an access sheath can be used to deliver the cardiac valve surgically (e.g., via thoracotomy or sternotomy).

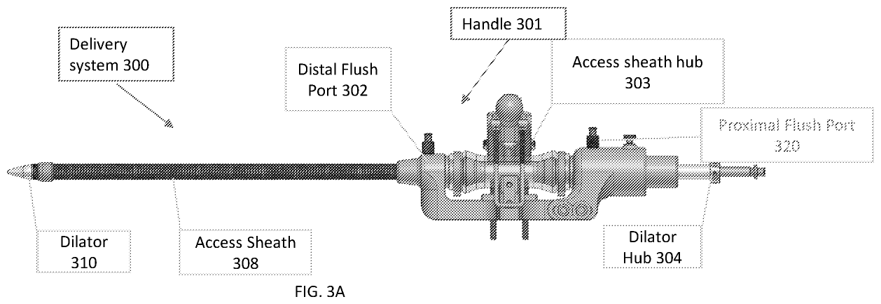

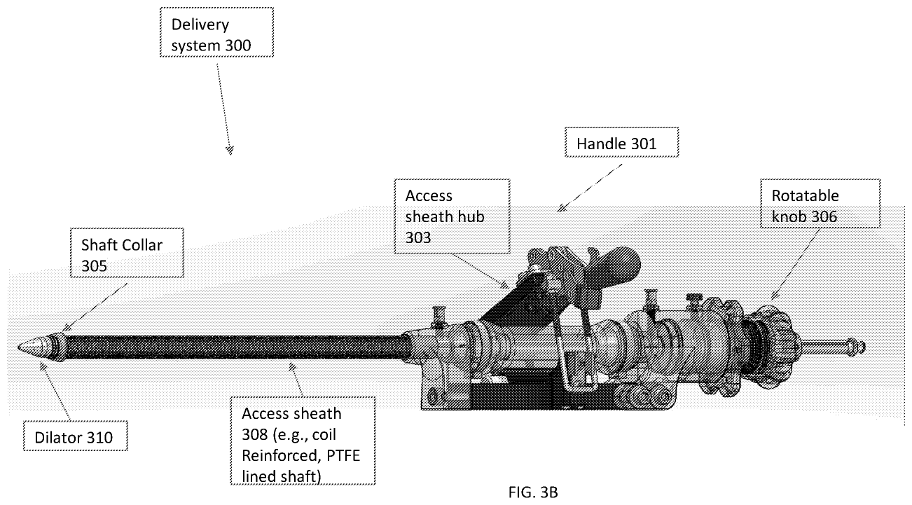

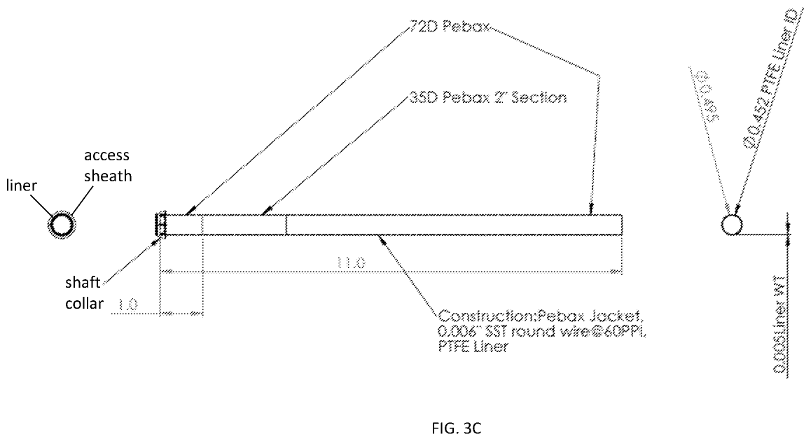

Referring to FIGS. 3A-3B, an access sheath 308 and inner dilator 310 can be part of an exemplary surgical delivery system 300. The access sheath 308 can include an inner lumen that is configured to accommodate one or more catheters and/or guidewires. A shaft collar 305 can extend around the distal end of the access sheath 308. The shaft collar 305 can be used to anchor the access sheath 308 into the left atrium and/or can provide radiopaque markers for positioning. The delivery system 300 can have a proximal handle 301 that includes a distal flush port 302, a proximal flush port 320, an access sheath hub 303, a rotatable knob assembly 306 configured to actuate a large bore hemostatic seal, and a dilator hub 304. The rotatable knob assembly 306 can be configured to control an inner diameter of the large bore hemostatic seal to accommodate elongate devices (e.g., catheters and/or guidewires) having different outer diameter sizes while providing a hemostatic seal between an outer environment and the inner lumen of the access sheath 308.

FIG. 3C shows exemplary dimensions for the access sheath 308. In some cases, the access sheath is constructed of a thermoplastic elastomer material (e.g., polyether block amide) with reinforcing wire. In some cases, the inner surface of the access sheath is lined with an inner liner (e.g., comprising polytetrafluoroethylene “PTFE”).

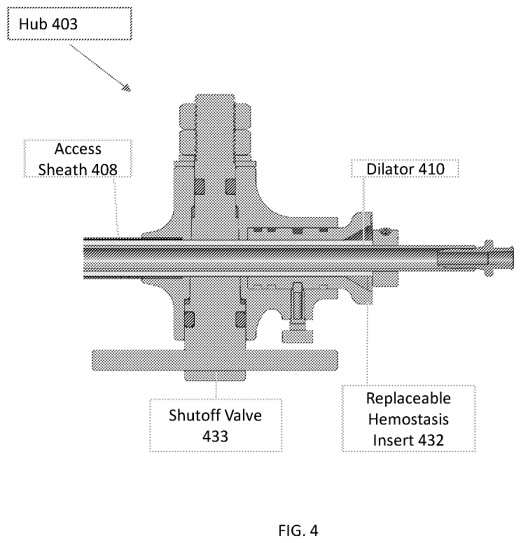

Referring to FIG. 4, in some embodiments, an access sheath 408 can include a hub 403 that is configured such that the access sheath 408 can maintain hemostasis with the dilator 410 and other components of the delivery system (including an anchor guide sheath, an anchor tether control shaft, an anchor tether, and/or a valve sheath) that are of varying sizes and/or are non-circular. Exemplary delivery device components are described in U.S. patent application Ser. No. 16/824,576, filed Mar. 19, 2020, and U.S. patent application Ser. No. 16/594,946, filed Oct. 7, 2019, the entire disclosures of which are incorporated by reference herein. To accommodate the components of differing sizes, the hub 403 can include a replaceable hemostasis insert 432, which can enable swappable or interchangeable hemostasis valves. A shut-off valve 433 can be placed distal of the hemostasis insert 432 to allow the swappable or interchangeable hemostasis valve to be removed and replaced while maintaining hemostasis, for example during a catheter exchange. For example, as shown in FIG. 4, the shut-off valve 433 can be configured as a stopcock wherein the stopcock is closed to swap out the hemostasis valve inserts.

FIGS. 5A-5B show another embodiment of a hub 503 having a replaceable hemostasis insert. The hub 503 can be adapted to controllably transition from an open configuration to a closed configuration to maintain hemostasis during a catheter exchange. Hub 503 is similar to hub 403 except that the shut-off valve 533 includes a pinch-clamp mechanism rather than a stopcock. The pinch-clamp mechanism can include compliant tubing that is configured to be pinched by the clamp to seal around various components (e.g., guidewires, anchor tether, etc.) regardless of their size, to maintain hemostasis.

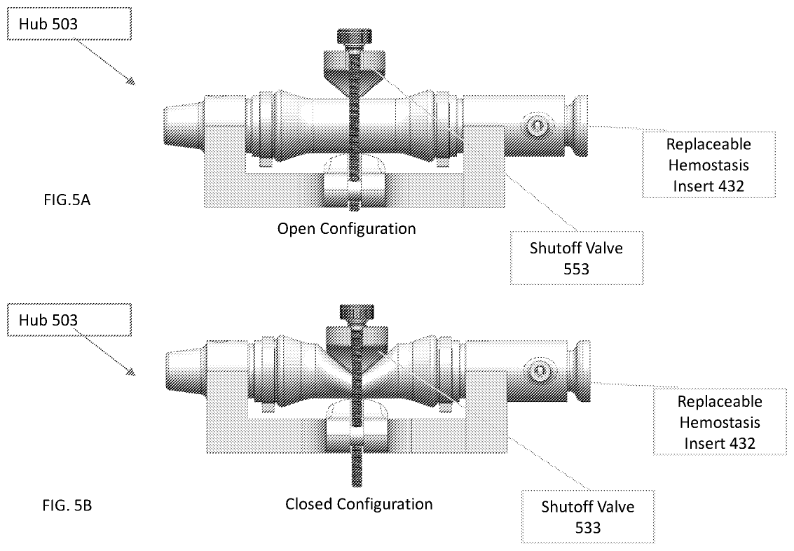

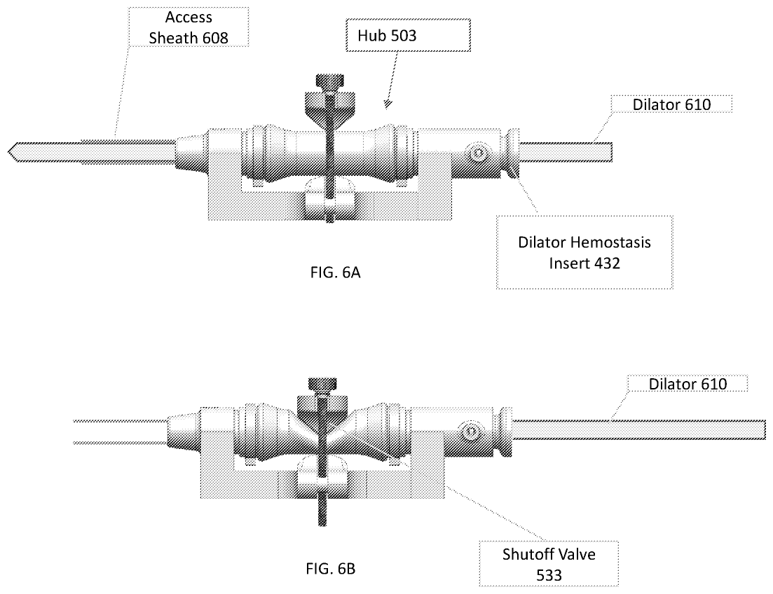

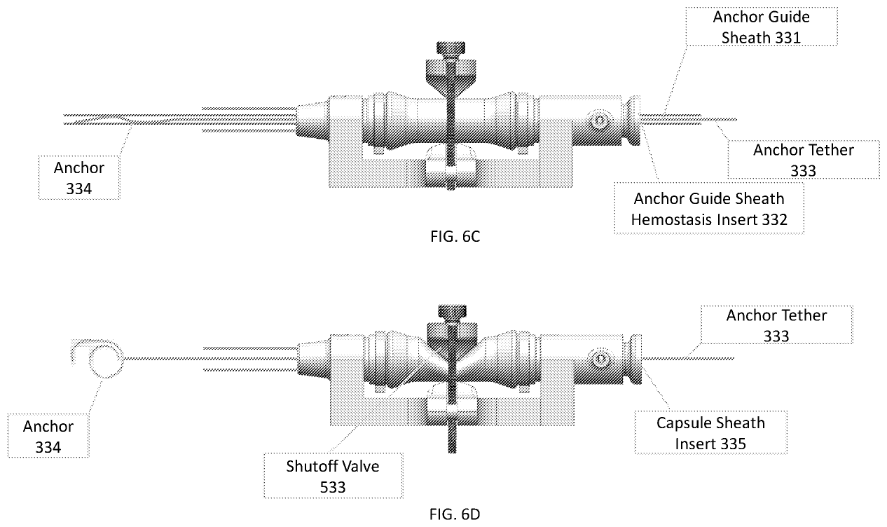

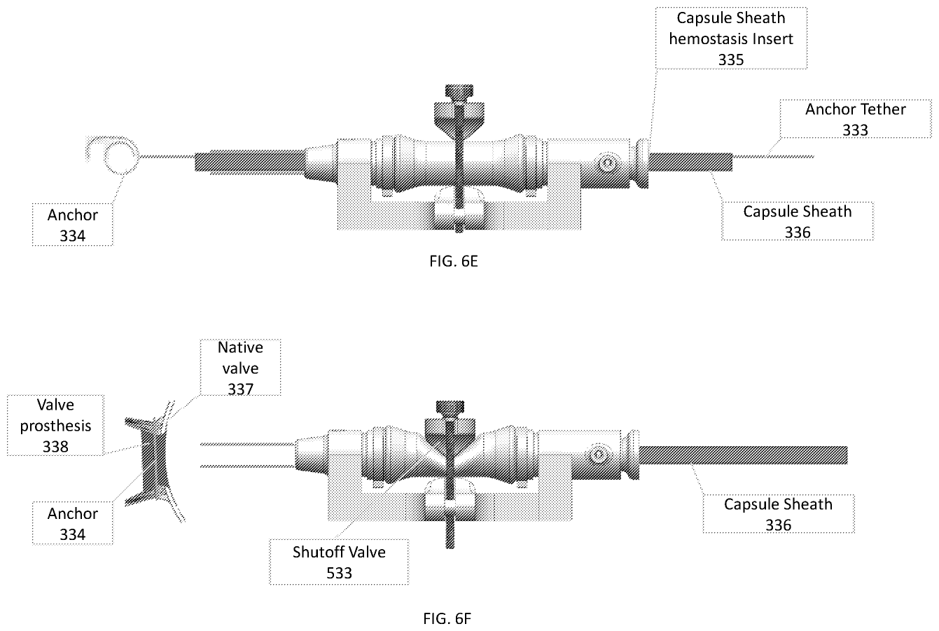

FIGS. 6A-6F show an exemplary use of the hub 503 with an access sheath 608 and dilator 610 in a trans-atrial (e.g., mitral) valve replacement procedure. Exemplary component dimensions for the procedure of FIGS. 6A-6F include: (1) access sheath dilator, 0.4375″ outer diameter (OD); (2) anchor guide sheath, 0.264″ outer diameter; (3) anchor tether control shaft, 0.065″ outer diameter; (4) anchor tether, 0.042″ outer diameter; (5) valve sheath, 0.427″ maj. OD×0.348″ min. OD. Advantageously, the hub 403 can maintain hemostasis with all of the variably-sized components. Although described with us in a trans-atrial procedure, the hub 503 can alternatively be used for trans-septal and/or surgical procedures. Hub 403 can similarly be used for trans-atrial, trans-septal, and/or surgical procedures.

FIG. 6A shows insertion of the access sheath 608 within the hub 503. FIG. 6B shows the dilator 610 retracted proximal to the shutoff valve but so that the tip remains in the hemostasis valve. The shutoff valve 533 is shut, then the dilator 610 is fully removed. FIG. 6C shows insertion of an anchor guide sheath 331 within an anchor guide sheath hemostasis insert 332. The anchor guide sheath 331 includes an anchor tether 333 and an anchor 334 housed therein. FIG. 6D shows the anchor guide sheath 331 retracted to deploy the anchor 334. The tip of the anchor guide sheath 331 remains in the hemostasis valve. The shutoff valve 533 is shut, then the anchor guide sheath 331 is fully removed. The shutoff valve 533 seals on the anchor tether 333 to allow swapping to a capsule sheath insert 335. FIG. 6E shows a capsule sheath 336 inserted into the hemostasis valve via the capsule sheath hemostasis insert 335. The shutoff valve 533 is opened, and the capsule sheath 336 is advanced into the atrium. FIG. 6F shows the valve prosthesis 338 implanted within the native valve 337 and within the spiral anchor 334. The anchor tether 333 is disconnected from the anchor 334 and removed. The capsule sheath 336 is retracted so that the tip remains in the hemostasis valve. The shutoff valve 533 is shut, then the capsule sheath 336 is fully removed.

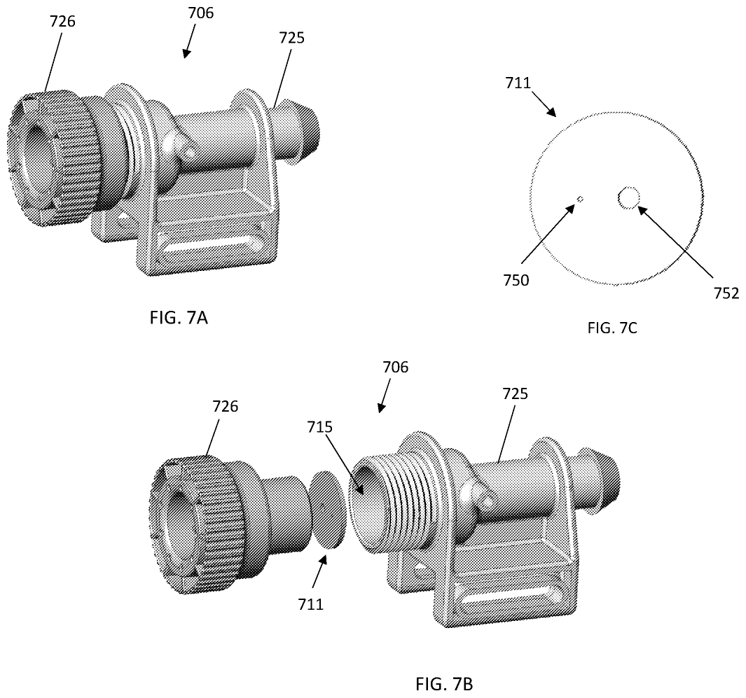

FIGS. 7A-7C show detailed views of a seal assembly 706 that can be used as an alternate to the rotatable knob assembly 306 of FIG. 3B, according to some embodiments. In contrast to the variable sizing achievable by the rotatable knob assembly 306, the seal assembly 706 can include components that accommodate elongate devices (e.g., catheters and/or guidewires) of predefined outer diameters. In some embodiments, seal assembly 706 includes a seal insert 711 having one or more apertures (openings) for passing and sealing about an elongate device. The seal insert 711 is sized and shaped to fit within a proximal opening 715 of a valve body 725. A distal end of a proximal nut 726 is configured to fit within the proximal opening of the valve body 725 to capture the seal insert 711 within the valve body 725. The proximal nut 726 can include a threaded interior that is configured to engage with a threaded exterior of the valve body 725 to secure the seal insert 711 therein.

As shown in FIG. 7C, an exemplary seal insert 711 has a disk shape and includes a first opening 750 and a second opening 752. The openings 750 and 752 can have diameters for accommodating respective elongate devices side-by-side and into the access sheath. The seal insert 711 can be made of a compliant material (e.g., silicone) to create a hemostatic seal between an outer environment and the inner lumen of the access sheath. The openings 750 and 752 can be spaced a distance apart to prevent the elongate devices from contacting each other within the seal assembly 706. The sizes of the openings 750 and 752 may vary depending on the sizes of the corresponding elongate devices. In the example shown, the first opening 750 is smaller than the second opening 752. Thus, the first opening 750 may be configured to accommodate a smaller elongate device than the second opening 752. For example, in some implementations, the first opening 750 may be configured to accommodate a first catheter or a guidewire, and the second opening 752 may be configured to accommodate a second catheter or a guidewire. In some cases, a catheter includes one or more catheters therein (nested catheters). Exemplary catheters used in some valve delivery procedures may include an anchor control catheter, a steerable guide catheter, and a valve delivery catheter.

The seal inserts described herein may include openings any size and are not limited to those shown in the exemplary seal insert 711. In some cases, the openings may be the same size. In addition, the seal insert 711 may include any number of openings (e.g., 1, 2, 3, 4, 5, 6 or more openings).

The seal assembly 706 can be configured so as to enable easy removal and replacement the seal insert 711 with one or more additional seal inserts during different stages and/or procedures of a medical intervention. For example, some medical interventions may require the use of a first catheter to perform a first procedure and a second catheter to perform a second procedure. In some cases, the first and second catheters may have different diameters. To accommodate differently sized catheters, the seal assembly 706 can include a first seal insert (e.g., 711) having an opening (e.g., 752) for accommodating the first catheter for the first procedure. After the first procedure is complete, the first catheter can be removed from the access sheath and the seal assembly 706. The proximal nut 726 may then be rotated to remove the first seal insert (e.g., 711) and be replaced with a second insert having an opening sized to accommodate the second catheter. After the second insert is secured within the seal assembly 706 (e.g., by reinserting/rethreading the proximal nut 726 into the valve body 725), the second catheter may be inserted within the seal assembly 706 and access sheath to perform the second procedure.

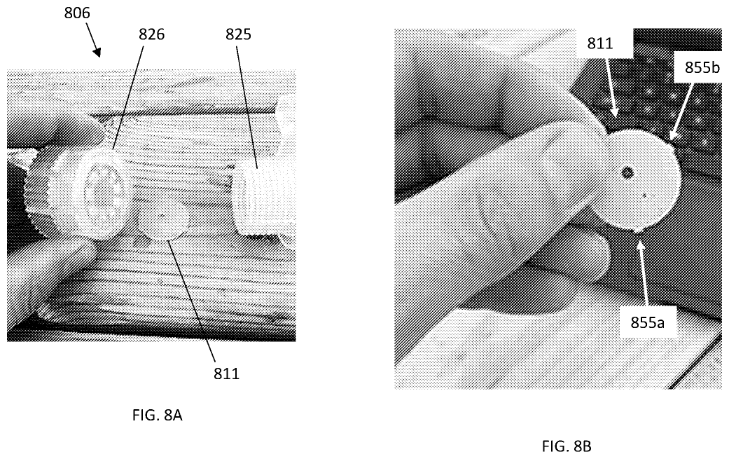

FIGS. 8A and 8B show a variation of the seal assembly 706 of FIGS. 7A-7C. As with the seal assembly 706, the seal assembly 806 includes a proximal nut 826 that is configured to threadably engage with a valve body 825 to secure a seal insert 811 within the valve body 825. In this example, the seal insert 811 includes alignment tabs 855a and 855b that are configured to mate with corresponding grooves within the valve body 825. When the seal insert 811 is properly secured within the valve body 825, the alignment tabs 855a and 855b are within the corresponding grooves, thereby locking a rotational orientation of the seal insert 811 with respect to the valve body 825. This alignment feature can assure that the various elongate devices are in a predetermined orientation with respect to the delivery system (e.g., 300) and/or the anatomy of the patient. The seal insert 811 can including any number of alignment tabs (e.g., 1, 2, 3, 4, 5, 6 or more alignment tabs).

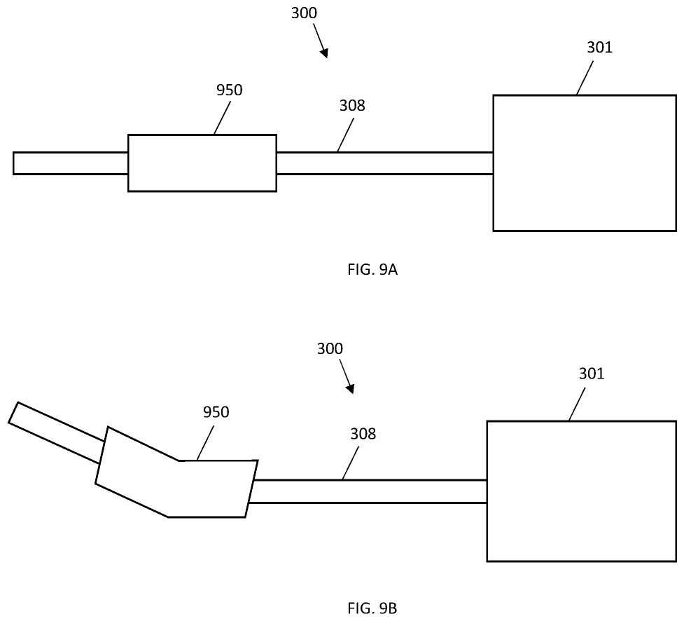

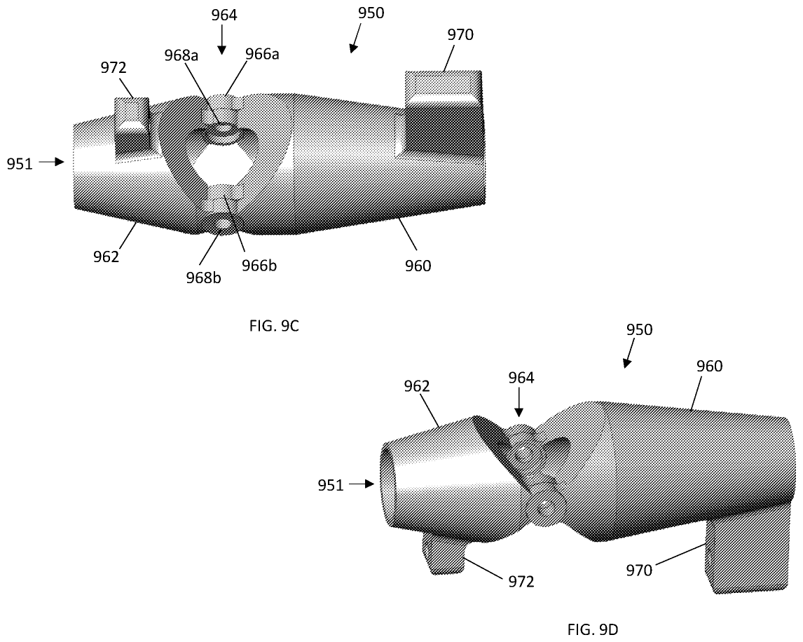

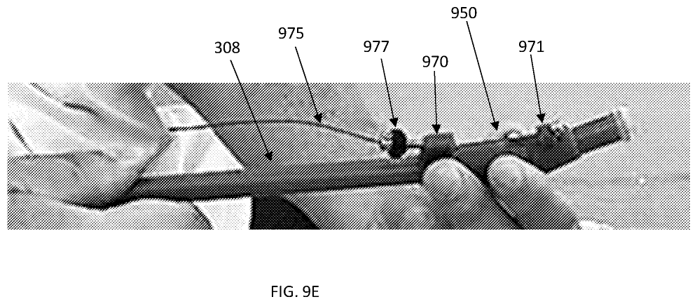

In some embodiments, the access sheaths described herein may be angled or bent in order to improve an entry angle into the patient's body. FIGS. 9A and 9B show a simple schematic of the delivery system 300 described above with the addition of a sheath bending component 950 that is configured to bend the access sheath 308. As shown, the sheath bending component 950 can be positioned around a circumference of the access sheath 308 at a location outside of the patient's body and distal to the handle 301. The sheath bending component 950 can be configured to transition between a first (e.g., straight) configuration, as shown in FIG. 9A, and a second (e.g., bent) configuration, as shown in FIG. 9B. The sheath bending component 950 can be configured to maintain the access sheath 308 in a straight state when in the straight configuration and maintain the access sheath 308 in a bent state when in the bent configuration. The sheath bending component 950 may be configured to bend the access sheath 308 to any extent to improve entry and maneuvering of the access sheath 308 in the patient's body.

FIGS. 9C and 9D shows a top view and a bottom view, respectively, of the exemplary sheath bending component 950. The sheath bending component 950 includes a central lumen 951 for accepting the access sheath. The sheath bending component 950 includes a proximal component 960 and a distal component 962 connected at a hinge 964. The proximal component 960 includes two openings 966a and 966b on opposing sides of the central lumen 951. Likewise, the distal component 962 includes two openings 968a and 968b on opposing sides of the central lumen 951. When the openings 966a and 966b of the proximal component 960 are aligned with the openings 968a and 968b of the distal component 962, bolts may be positioned therein to hingeably couple the proximal 960 and distal 962 components. Cable-holding portions 970 and 972 of the proximal component 960 and distal component 962, respectively, include holes for accepting a cable, which may be used to control the activation of the hinge 964.

FIG. 9E shows an exemplary view of an access sheath 308 being bent using the sheath bending component 950. A cable 975 is threaded through the holes of the cable-holding portions 970 and 972 of the sheath bending component 950. A control 977 (e.g., knob) is coupled to and configured to control tension the cable 975. Increasing tension on the cable 975 causes the sheath bending component 950 to bend at the hinge 964, thereby causing the access sheath 308 to bend correspondingly.

Further, although described for use in a mitral valve replacement procedure, it should be understood that the access sheaths and hubs described herein could be used for a variety of medical procedures. For example, the access sheaths and hubs (e.g., including the interchangeable inserts and shut-off valves) can be used in any trans-catheter structural heart delivery system (such as for delivery of LAA occluders, aortic valves, mitral repair systems, and shunts). Similarly, the access sheaths and hubs can be used for delivery of neurological devices, including aneurysm coils, aspiration systems, and mechanical thrombectomy systems.

It should be understood that any feature described herein with respect to one embodiment can be used in addition to or in place of any feature described with respect to another embodiment.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A delivery system for delivering a valve prosthesis, the delivery system comprising:
one or more catheters holding at least a portion of the valve prosthesis therein;
an access sheath having an inner lumen for accommodating the one or more catheters; and
a handle coupled to a proximal end of the access sheath, the handle comprising a seal configured to accommodate the one or more catheters therethrough and to provide a hemostatic seal between an outer environment and the inner lumen of the access sheath.

2. The delivery system of claim 1, wherein the seal is a disk-shaped insert having a first opening and a second opening, wherein the first opening has a first diameter that is configured to accept and seal with a first catheter, wherein the second opening has a second diameter that is configured to accept and seal with a second catheter.

3. A delivery system for delivering a valve prosthesis, the delivery system comprising:
one or more catheters holding at least a portion of the valve prosthesis therein;
an access sheath having an inner lumen for accommodating the one or more catheters;
a handle coupled to a proximal end of the access sheath, the handle configured to provide access for the one or more catheters distally through the access sheath; and
a sheath bending component positioned around a circumference of the access sheath, the sheath bending component configured to controllably bend the access sheath.

4. The delivery system of claim 3, wherein the sheath bending component includes a hinge and a cable, wherein tensioning of the cable causes the hinge to bend, thereby causing the access sheath to bend accordingly.
